(19) United States
(12) Patent Application Publication
(10) Pub. No.: US 2022/0284790 A1
(43) Pub. Date: Sep. 8, 2022

(54) POSTURE DISCRIMINATION DEVICE

(71) Applicant: AISIN CORPORATION, Kariya (JP)

(72) Inventor: Noriyuki NISHIZAWA, Kariya-shi (JP)

(73) Assignee: AISIN CORPORATION, Kariya (JP)

(21) Appl. No.: 17/665,700

(22) Filed: Feb. 7, 2022

(30) Foreign Application Priority Data
Mar. 2, 2021 (JP) 2021-032574
Dec. 2, 2021 (JP) 2021-196171

(51) Int. Cl. G08B 21/04; G06T 7/73

(52) U.S. Cl. CPC G08B 21/0476; G08B 21/043; G06T 7/73; G06T 2207/30196

(57) ABSTRACT

A posture discrimination device includes: a skeleton point detection unit detecting a skeleton point of a person in an image; a posture discrimination probability value calculation unit calculating a posture discrimination probability value of the person based on detection of the skeleton point; and a posture discrimination unit discriminating a posture of the person based on the posture discrimination probability value. The posture discrimination probability value calculation unit calculates a falling discrimination probability value indicating a probability of the posture of the person being a falling posture. The posture discrimination unit includes a falling erroneous discrimination determination unit determining an erroneous discrimination of the falling posture based on the falling discrimination probability value. The falling erroneous discrimination determination unit determines that there is a possibility of the erroneous discrimination when a change speed of the falling discrimination probability value is equal to or higher than a speed threshold value.

### CROSS REFERENCE TO RELATED APPLICATIONS

This application is based on and claims priority under 35 U.S.C. § 119 to Japanese Patent Applications 2021-32574 and 2021-196171, filed on Mar. 2, 2021 and December 2, 2021, respectively, the entire content of which is incorporated herein by reference.

### TECHNICAL FIELD

This disclosure relates to a posture discrimination device.

### BACKGROUND DISCUSSION

In the related art, a method for discriminating, by detecting a skeleton point of a person included in a captured image, a posture of the imaged person has been proposed. For example, a posture detection device described in WO2016/143641 (Reference 1) detects a head and a body trunk of a person as skeleton points shown in a captured image. In this posture detection device, a position, size, orientation, and the like of the head are detected. Further, a positional relationship between the head and the body trunk is detected. Then, based on these parameters, the posture of the person shown in the captured image is discriminated.

However, when the skeleton points of the person included in the captured image are detected, a detection accuracy of the skeleton point may decrease depending on a positional relationship between the person and a camera. Therefore, there is a possibility that the posture of the person is erroneously discriminated.

### SUMMARY

A posture discrimination device includes: a skeleton point detection unit configured to detect a skeleton point of a person included in a captured image; a posture discrimination probability value calculation unit configured to calculate a posture discrimination probability value of the person based on detection of the skeleton point; and a posture discrimination unit configured to discriminate a posture of the person based on the posture discrimination probability value. The posture discrimination probability value calculation unit calculates, as the posture discrimination probability value, a falling discrimination probability value indicating a probability that the posture of the person is a falling posture. The posture discrimination unit includes a falling erroneous discrimination determination unit configured to determine an erroneous discrimination of the falling posture based on the falling discrimination probability value. The falling erroneous discrimination determination unit determines that there is a possibility of occurrence of the erroneous discrimination when a change speed of the falling discrimination probability value is equal to or higher than a predetermined speed threshold value.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a perspective view of a vehicle provided with a posture discrimination device;

FIG. 2 is an explanatory view of an occupant in a vehicle cabin and a camera for capturing an image of the occupant;

FIG. 3 is a block view showing a schematic configuration of a control device that functions as the posture discrimination device;

FIG. 4 is an explanatory view showing skeleton points of a person;

FIG. 5 is a flowchart showing a processing procedure of a posture discrimination based on detection of the skeleton points;

FIG. 6 is a flowchart showing a processing procedure of a falling erroneous discrimination determination based on a change speed of a falling discrimination probability value;

FIG. 7 is a flowchart showing a processing procedure of a falling erroneous discrimination determination based on height of the skeleton points;

FIG. 8 is a flowchart showing a processing procedure for confirming an erroneous discrimination of a falling posture based on a possibility of occurrence of the erroneous discrimination; and

FIG. 9 is a flowchart showing a mode of a processing at a time of a falling erroneous discrimination.

### DETAILED DESCRIPTION

Hereinafter, an embodiment of a posture discrimination device will be described with reference to drawings.

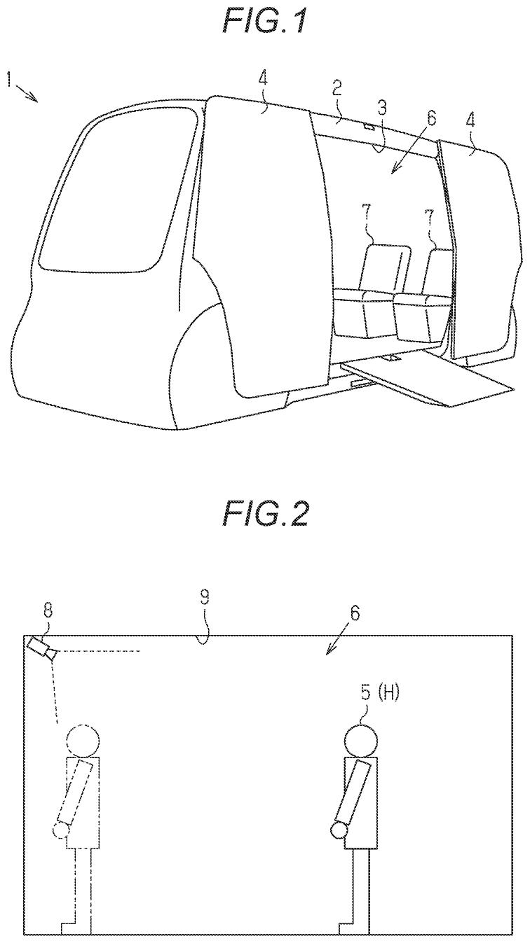

As shown in FIGS. 1 and 2, a vehicle 1 according to the present embodiment has a substantially rectangular box-shaped vehicle body 2 extending in a front-rear direction of the vehicle. In addition, a door opening 3, which serves as an entrance and exit of an occupant, is provided on a side surface of the vehicle body 2. The door opening 3 is provided with a pair of slide doors 4, 4 that open and close in the front-rear direction and opposite directions of the vehicle. An occupant 5 in the vehicle 1 rides on the vehicle 1 in a “sitting posture” in which the occupant 5 sits on one of seats 7 provided in a vehicle cabin 6, or in a “standing posture” in which the occupant 5 stands using, for example, a hanging strap or a handrail (not shown).

In addition, the vehicle 1 according to the present embodiment is provided with a camera 8 for capturing an image of the inside of the vehicle cabin 6. In the vehicle 1 according to the present embodiment, the camera 8 is provided on a ceiling portion 9 of the vehicle cabin 6. For example, a visible light camera or the like is used as the camera 8. The camera 8 according to the present embodiment captures an image of a person H in the vehicle cabin 6, that is, the occupant 5 who rides on the vehicle 1, from above.

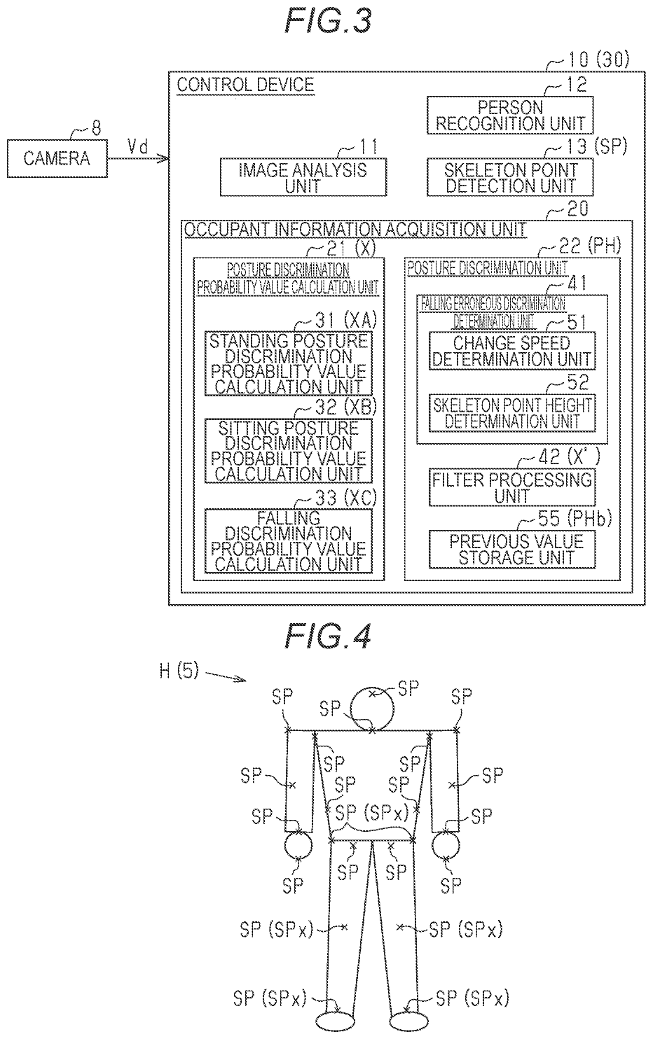

As shown in FIG. 3, in the vehicle 1 according to the present embodiment, a captured image Vd in the vehicle cabin 6 captured by the camera 8 is input to a control device 10. The control device 10 according to the present embodiment includes an image analysis unit 11 that analyzes the captured image Vd, and a person recognition unit 12 that recognizes the person H in the vehicle cabin 6 shown in the captured image Vd, that is, the occupant 5 of the vehicle 1, based on a result of the image analysis, in cooperation with the image analysis unit 11.

Further, as shown in FIGS. 3 and 4, the control device 10 according to the present embodiment includes a skeleton point detection unit 13 that detects skeleton points SP of the person H included in the captured image Vd, based on the result of the image analysis, in cooperation with the image analysis unit 11 and the person recognition unit 12. That is, the skeleton points SP are unique points characterizing a body of the person H, such as joints or points on a body surface, and correspond to, for example, head, neck, shoulders, armpits, elbows, wrists, fingers, waist, hip joints, buttocks, knees, and ankles. Further, the control device 10 according to the present embodiment includes an occupant information acquisition unit 20 that acquires information on the occupant 5 shown in the captured image Vd based on the detection of the skeleton points SP by the skeleton point detection unit 13.

Specifically, the person recognition unit 12 according to the present embodiment uses an inference model generated by machine learning to execute a recognition processing of the person H. Then, the skeleton point detection unit 13 also uses the inference model generated by the machine learning to execute a detection processing of the skeleton points SP.

In addition, the occupant information acquisition unit 20 includes a posture discrimination probability value calculation unit 21 that calculates a posture discrimination probability value X of the person H shown in the captured image Vd based on the detection of the skeleton points SP, and a posture discrimination unit 22 that discriminates a posture PH of the person H using the posture discrimination probability value X. Then, the control device 10 according to the present embodiment has a function as a posture discrimination device 30.

More specifically, the posture discrimination probability value calculation unit 21 according to the present embodiment calculates feature amounts based on positions of the skeleton points SP such as the head and the shoulders of the occupant 5 detected from the captured image Vd. Specifically, the posture discrimination probability value calculation unit 21 according to the present embodiment calculates feature amounts based on positions in two-dimensional coordinates in the captured image Vd. In addition, the posture discrimination probability value calculation unit 21 calculates feature amounts based on body dimensions indicated by a plurality of skeleton points SP, such as a shoulder width of the occupant 5. Then, the posture discrimination probability value calculation unit 21 according to the present embodiment calculates the posture discrimination probability value X by inputting these feature amounts into the inference model generated by the machine learning.

More specifically, the posture discrimination probability value calculation unit 21 according to the present embodiment includes a standing posture discrimination probability value calculation unit 31 that calculates a probability that the posture PH of the person H shown in the captured image Vd in the vehicle cabin 6, that is, the posture PH of the occupant 5 serving as a target person of a posture determination is the “standing posture”. In addition, the posture discrimination probability value calculation unit 21 includes a sitting posture discrimination probability value calculation unit 32 that calculates a probability that the posture PH of the occupant 5 serving as the target person is the “sitting posture”. In addition, the posture discrimination probability value calculation unit 21 according to the present embodiment includes a falling discrimination probability value calculation unit 33 that calculates a probability that the posture PH of the occupant 5 serving as the target person is the “falling posture”.

That is, the posture discrimination unit 22 according to the present embodiment acquires, as the posture discrimination probability values X, a standing posture discrimination probability value XA calculated by the standing posture discrimination probability value calculation unit 31, a sitting posture discrimination probability value XB calculated by the sitting posture discrimination probability value calculation unit 32, and a falling discrimination probability value XC calculated by the falling discrimination probability value calculation unit 33. Further, the posture determination probability value calculation unit 21 calculates the posture discrimination probability values X such that a total value of the standing posture discrimination probability value XA, the sitting posture discrimination probability value XB, and the falling discrimination probability value XC is “1.0”. Therefore, the posture discrimination unit 22 according to the present embodiment can discriminate the posture PH of the occupant 5 without any contradiction based on the posture discrimination probability values X.

The standing posture discrimination probability value XA calculated by the standing posture discrimination probability value calculation unit 31 according to the present embodiment is further divided into a probability that the occupant 5 in the “standing posture” is in a “moving state”, a probability that the occupant 5 is in a “still state”, and a probability that the occupant 5 is in a “state of standing by using the hanging strap, the handrail, and the like”. Then, the posture discrimination unit 22 according to the present embodiment can subdivide and discriminate the “standing posture”.

### Falling Erroneous Discrimination Determination

Next, in the control device 10 according to the present embodiment, a falling erroneous discrimination determination executed by the posture discrimination unit 22 and a mode of a posture discrimination processing based on a determination result will be described.

As shown in FIG. 3, the control device 10 according to the present embodiment includes a falling erroneous discrimination determination unit 41 provided in the posture discrimination unit 22. In the posture discrimination unit 22 according to the present embodiment, the falling erroneous discrimination determination unit 41 determines an erroneous discrimination of the “falling posture” based on the falling discrimination probability value XC. Then, the posture discrimination unit 22 according to the present embodiment changes the mode of the posture discrimination processing based on the determination result by the falling erroneous discrimination determination unit 41.

That is, as shown in FIG. 2, in the vehicle 1 according to the present embodiment, when the occupant 5 in the vehicle cabin 6 moves below the camera 8 provided in the ceiling portion 9, an image of the occupant 5 shown in the captured image Vd is likely to be in a state in which a lower body is hidden by the head or an upper body of the occupant 5. Further, a detection accuracy of the skeleton points SP is lowered, and thus a calculation accuracy of the posture discrimination probability value X is also lowered. For example, since the skeleton points SP of the person H included in the captured image Vd are densely detected, the falling discrimination probability value XC may increase. As a result, in the control device 10 according to the present embodiment, in such a case, there is a problem that the erroneous discrimination is likely to occur in the discrimination of the “falling posture” based on the falling discrimination probability value XC.

In view of this point, in the control device 10 according to the present embodiment, as described above, the falling erroneous discrimination determination unit 41 provided in the posture discrimination unit 22 determines the erroneous discrimination of the “falling posture” based on the falling discrimination probability value XC. Therefore, the control device 10 according to the present embodiment can accurately discriminate the posture PH of the occupant 5 shown in the captured image Vd by suppressing the erroneous discrimination of the “falling posture” from being a final output of a posture discrimination.

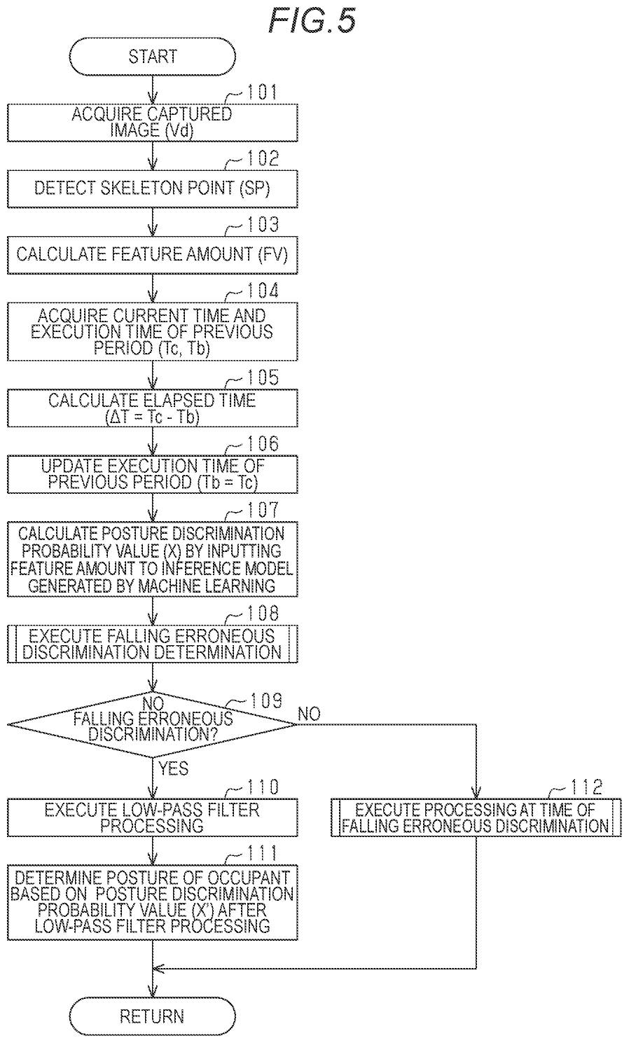

More specifically, as shown in a flowchart of FIG. 5, in the control device 10 according to the present embodiment, the captured image Vd in the vehicle cabin 6 captured by the camera 8 is periodically acquired (step 101). Subsequently, the skeleton point detection unit 13 detects the skeleton points SP (step 102), and the posture discrimination probability value calculation unit 21 calculates feature amounts FV based on the detection of the skeleton points SP (step 103). In the control device 10 according to the present embodiment, a current time Tc and an execution time Tb of a previous period are acquired at this timing (step 104), and an elapsed time ΔT from the previous period to a current period is calculated (ΔT=Tc−Tb, step 105). Further, the current time Tc calculated in step 103 updates the execution time Tb of the previous period stored in a storage area (not shown) (Tb=Tc, step 106). Then, the posture discrimination probability value calculation unit 21 calculates the posture discrimination probability value X by inputting the feature amounts FV calculated in step 103 to the inference model generated by the machine learning (step 107).

In the control device 10 according to the present embodiment, next, the falling erroneous discrimination determination unit 41 executes the falling erroneous discrimination determination (step 108). Further, the posture discrimination unit 22 determines whether the determination result by the falling erroneous discrimination determination unit 41 indicates the erroneous discrimination of the “falling posture” based on the falling discrimination probability value XC (step 109). Then, when the determination result of the falling erroneous discrimination determination unit 41 is “no erroneous discrimination” (step 109: YES), the posture discrimination unit 22 according to the present embodiment executes a posture discrimination processing in a normal state based on the posture discrimination probability value X calculated in the current period.

Specifically, as shown in FIG. 3, the posture discrimination unit 22 according to the present embodiment includes a filter processing unit 42 that executes a low-pass filter processing on the posture discrimination probability value X calculated by the falling discrimination probability value calculation unit 33. Then, the low-pass filter processing executed by the filter processing unit 42 according to the present embodiment can be expressed by a following Formula (1).

$$y [ n ] = {\sum }_{i = 0}^{P} {b}_{j} x [ n - i ] - {\sum }_{j = 1}^{Q} {a}_{j} y [ n - j ] \qquad ( 1 )$$

However, n: current, y: a filter value of the posture discrimination probability value, x: an input value of the posture discrimination probability value, b, a: coefficient, and P, Q: the number of times of filter.

That is, in the posture discrimination unit 22 according to the present embodiment, the low-pass filter processing executed by the filter processing unit 42 reflects a history of the posture discrimination probability value X calculated periodically in synchronization with the detection of the skeleton points SR That is, a value of the posture discrimination probability value X calculated in or before the previous period is used.

As shown in FIG. 5, the posture discrimination unit 22 according to the present embodiment first executes this low-pass filter processing for the posture discrimination probability value X calculated in step 107 as the posture discrimination processing in the normal state (step 110). Further, the posture discrimination unit 22 determines a posture of the occupant 5 serving as the target person based on a posture discrimination probability value X′ after the low-pass filter processing (step 111). Therefore, the posture discrimination unit 22 according to the present embodiment is configured such that a blur such as chattering is less likely to occur in a result of the posture discrimination based on the posture discrimination probability value X.

On the other hand, when the determination result of the falling erroneous discrimination determination unit 41 is the “erroneous discrimination” in step 109 (step 109: NO), the posture discrimination unit 22 according to the present embodiment does not execute processes of step 110 and step 111. Then, the posture discrimination unit 22 executes a processing at a time of falling erroneous discrimination corresponding to a case where the erroneous discrimination of the “falling posture” is determined (step 112).

More specifically, as shown in FIG. 3, the falling erroneous discrimination determination unit 41 according to the present embodiment includes a change speed determination unit 51 that determines, based on a change speed of the falling discrimination probability value XC, a possibility of occurrence of the erroneous discrimination in the discrimination of the “falling posture” based on the falling discrimination probability value XC.

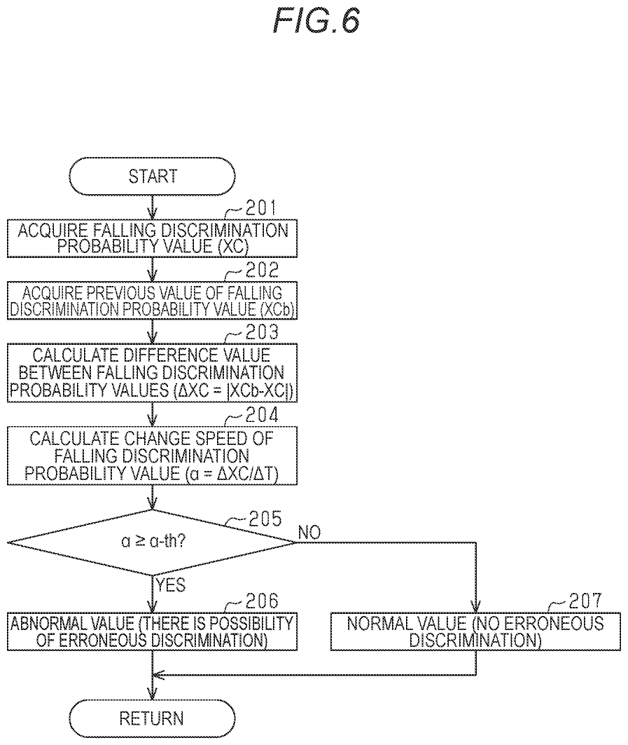

Specifically, as shown in a flowchart of FIG. 6, the change speed determination unit 51 according to the present embodiment first acquires the falling discrimination probability value XC calculated in the current period (step 201). Next, when the change speed determination unit 51 acquires a previous value XCb of the falling discrimination probability value calculated in the previous period (step 202), the change speed determination unit 51 subsequently calculates a difference value ΔXC between the previous value XCb and the current falling discrimination probability value XC (ΔXC=|XCb−XC|, step 203). In the control device 10 according to the present embodiment, the previous value XCb of the falling discrimination probability value is stored in the storage area (not shown). Then, the change speed determination unit 51 according to the present embodiment calculates a change speed α of the falling discrimination probability value XC by dividing the difference value ΔXC calculated in step 103 by the elapsed time ΔT from the previous period to the current period (α=αXC/αT, step 204).

Next, the change speed determination unit 51 according to the present embodiment compares the change speed α of the falling discrimination probability value XC with a predetermined speed threshold value α-th (step 205). Then, when the change speed α is equal to or higher than the speed threshold value α-th (α≥α-th, step 205: YES), it is determined that the erroneous discrimination may occur in the discrimination of the “falling posture” based on the falling discrimination probability value XC calculated in the current period (step 206).

That is, it takes some time for the posture PH of the person H to change from the “standing posture” to the “falling posture”. For example, a following reference document describes that when the walking person H falls, it takes about 0.3 seconds for the knees of the person H to touch the ground. Then, a maximum speed when a time required for this fall is converted into the change speed α of the falling discrimination probability value XC is “3.3/second”.

(Reference Document) “E. T. Hsiao and S. N. Robinovitch, “Common protective movements govern unexpected falls from standing height.” Journal of biomechanics, vol. 31, no. 1, pp. 1-9, 1997”.

In view of this point, in the change speed determination unit 51 according to the present embodiment, when the occupant 5 in the vehicle cabin 6 shown in the captured image Vd actually falls, the speed threshold value α-th is set corresponding to an assumed value generated at the change speed α of the falling discrimination probability value XC. Then, when the change speed α equal to or greater than the speed threshold value α-th is generated, the change speed determination unit 51 according to the present embodiment determines that the erroneous discrimination of the “falling posture” may occur with the change speed α as an abnormal value.

In step 205, when the change speed α of the falling discrimination probability value XC is lower than the speed threshold value α-th (α<α-th, step 205: NO), it is determined that this speed threshold value α-th is a normal value. Then, the change speed determination unit 51 according to the present embodiment determines that there is no erroneous discrimination in the discrimination of the “falling posture” based on the falling discrimination probability value XC calculated in the current period (step 207).

In addition, as shown in FIG. 3, the falling erroneous discrimination determination unit 41 according to the present embodiment includes a skeleton point height determination unit 52 that determines, based on a height β of the skeleton points SP shown in the captured image Vd, the possibility of occurrence of the erroneous discrimination in the discrimination of the “falling posture” based on the falling discrimination probability value XC.

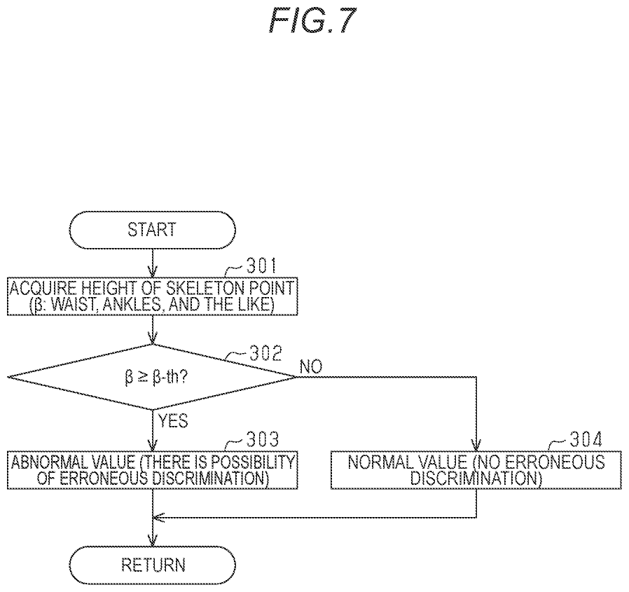

More specifically, as shown in a flowchart of FIG. 7, the skeleton point height determination unit 52 according to the present embodiment first acquires the height β of the skeleton points SP from the captured image Vd of the occupant 5 serving as the target person (step 301).

Specifically, as shown in FIG. 4, in the skeleton point height determination unit 52 according to the present embodiment, predetermined skeleton points SPx are set in advance as the skeleton points SP for acquiring the height β in the captured image Vd. Specifically, for example, the skeleton points SP located in the lower body of the occupant 5, such as the waist, the knees, or the ankles, are used for the predetermined skeleton points SPx. In addition, in the vehicle 1 according to the present embodiment, a Y-axis direction of the two-dimensional coordinates (X, Y) in the captured image Vd captured by the camera 8 is set to be a height direction of the vehicle cabin 6. In other words, in the posture discrimination device 30 configured by the control device 10 according to the present embodiment, a Y axis of the captured image Vd is a height coordinate. Then, the skeleton point height determination unit 52 according to the present embodiment acquires the height β of the skeleton points SP shown in a Y-axis coordinate in the captured image Vd.

As shown in FIG. 7, the skeleton point height determination unit 52 according to the present embodiment next compares the height β of the skeleton points SP acquired from the captured image Vd with a predetermined height threshold value β-th (step 302). Then, when the height β of the skeleton points SP is equal to or greater than the height threshold value β-th (β≥β-th, step 302: YES), it is determined that the erroneous discrimination may occur in the discrimination of the “falling posture” based on the falling discrimination probability value XC calculated in the current period (step 303).

That is, when the posture PH of the person H changes from the “standing posture” to the “falling posture”, the height β of the skeleton points SP shown in the captured image Vd becomes low. In view of this point, in the skeleton point height determination unit 52 according to the present embodiment, the height threshold value β-th is set for the predetermined skeleton points SPx corresponding to the assumed value when the occupant 5 in the vehicle cabin 6 shown in the captured image Vd actually falls. Then, when the acquired height β of the skeleton points SP is equal to or greater than the height threshold value β-th, the skeleton point height determination unit 52 according to the present embodiment determines that the erroneous discrimination of the “falling posture” may occur with a value of the height β as an abnormal value.

In step 302, when the height β of the skeleton points SP is lower than the height threshold value β-th (β<β-th, step 302: NO), the height threshold value β-th is determined to be a normal value. Then, the skeleton point height determination unit 52 according to the present embodiment determines that there is no erroneous discrimination in the discrimination of the “falling posture” based on the falling discrimination probability value XC calculated in the current period (step 304).

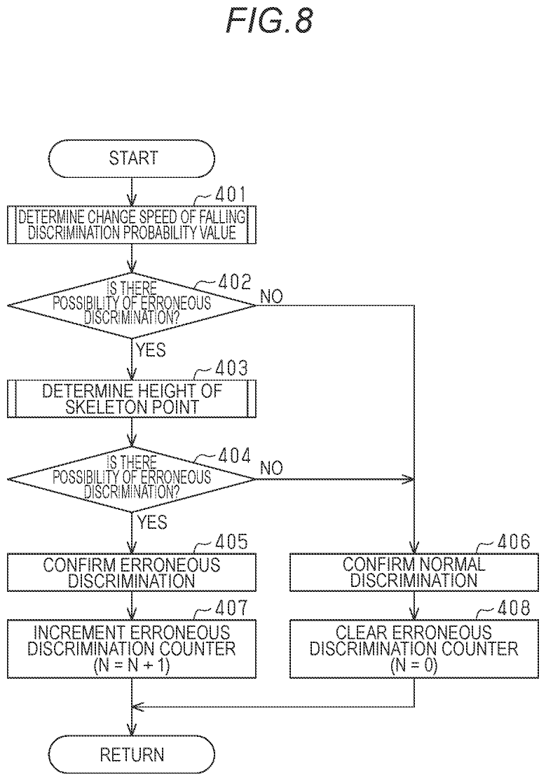

More specifically, as shown in a flowchart of FIG. 8, in the falling erroneous discrimination determination unit 41 according to the present embodiment, first, the change speed determination unit 51 determines the change speed of the falling discrimination probability value XC (step 401). In addition, when it is determined that the erroneous discrimination of the “falling posture” may occur in the determination of the change speed of the falling discrimination probability value XC (step 402: YES), subsequently, the height of the skeleton points SP is determined by the skeleton point height determination unit 52 (step 403). Then, when the falling erroneous discrimination determination unit 41 according to the present embodiment determines that the erroneous discrimination of the “falling posture” may occur in the height determination of the skeleton points SP (step 404: YES), the falling erroneous discrimination determination unit 41 confirms the erroneous discrimination of the “falling posture” based on the falling discrimination probability value XC (step 405).

In addition, when the erroneous discrimination of the “falling posture” is denied based on the change speed α of the falling discrimination probability value XC (step 402: NO), the falling erroneous discrimination determination unit 41 confirms a normal discrimination of the “falling posture” based on the falling discrimination probability value XC (step 406). Even when the erroneous discrimination of the “falling posture” is denied based on the height β of the skeleton points SP (step 404: NO), the falling erroneous discrimination determination unit 41 according to the present embodiment confirms the normal discrimination of the “falling posture” based on the falling discrimination probability value XC in step 406.

Further, the falling erroneous discrimination determination unit 41 according to the present embodiment increments a counter (not shown) when the erroneous discrimination of the “falling posture” is confirmed in step 405 (N=N+1, step 407). On the other hand, when the normal discrimination of the “falling posture” is confirmed in step 406, the counter (not shown) is cleared (N=0, step 408). That is, the falling erroneous discrimination determination unit 41 according to the present embodiment has a function of counting the number of consecutive times N of the periods in which the erroneous discrimination of the “falling posture” is confirmed. The posture discrimination unit 22 according to the present embodiment executes the processing at the time of the falling erroneous discrimination corresponding to a case where the erroneous discrimination of the “falling posture” is determined based on the number of consecutive times N of the periods in which the erroneous discrimination is confirmed (see FIG. 5, step 112).

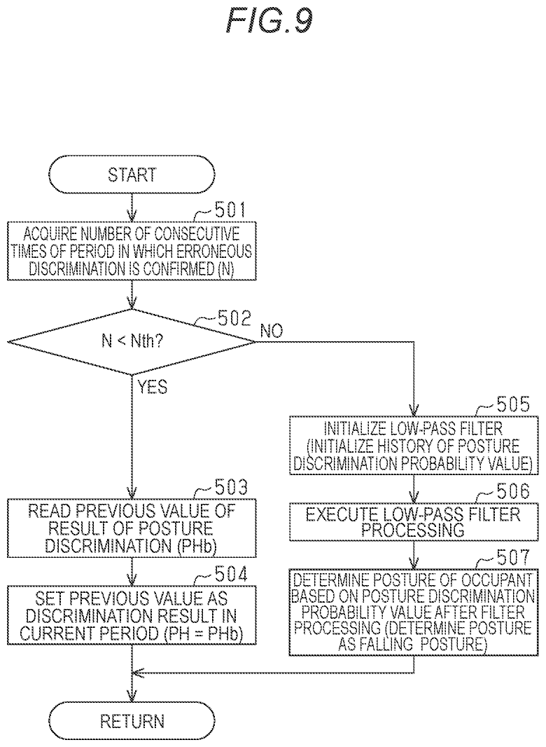

More specifically, as shown in FIG. 9, when the posture discrimination unit 22 according to the present embodiment acquires the number of consecutive times N of the periods in which the erroneous discrimination of the “falling posture” is confirmed (step 501), the posture discrimination unit 22 compares the number of consecutive times N with a predetermined consecutive threshold value Nth (step 502). In the posture discrimination unit 22 according to the present embodiment, the consecutive threshold value Nth is set to, for example, about “3 times”. When the number of consecutive times N is less than the consecutive threshold value Nth, the posture PH of the occupant 5 is determined so that the erroneous discrimination of the “falling posture” based on the falling discrimination probability value XC calculated in the current period is not the final output of the posture discrimination.

Specifically, as shown in FIG. 3, the posture discrimination unit 22 according to the present embodiment includes a previous value storage unit 55 that stores the posture PH of the occupant 5 discriminated in the previous period, that is, the result of the posture discrimination in the previous period as a previous value PHb. As described above, when the erroneous discrimination of the “falling posture” is confirmed in the falling erroneous discrimination determination unit 41 (see FIG. 8, step 405), the previous value PHb is used as the result of the posture discrimination in the current period.

That is, as shown in FIG. 9, the posture discrimination unit 22 according to the present embodiment reads the previous value PHb from the previous value storage unit 55 when the number of consecutive times N of the erroneous discrimination is less than the consecutive threshold value Nth (N<Nth, step 502: YES) when the processing at the time of the falling erroneous discrimination is executed (step 503). In step 504 following step 503, the result of the posture discrimination in the previous period shown in the previous value PHb is used for the posture discrimination in the current period. That is, the posture PH of the occupant 5 discriminated in the previous period is set to the posture PH of the occupant 5 discriminated in the current period (PH=PHb, step 504).

In addition, in step 502, when the number of consecutive times N of the erroneous discrimination reaches the consecutive threshold value Nth (N≥Nth, step 502: NO), the posture discrimination unit 22 according to the present embodiment does not execute processes of step 503 and step 504. The posture discrimination unit 22 according to the present embodiment discriminates that the posture PH of the occupant 5 is the “falling posture” regardless of the determination result of the falling erroneous discrimination determination unit 41 that confirms the erroneous discrimination of the “falling posture” (see FIG. 8, step 405).

More specifically, in this case, the posture discrimination unit 22 according to the present embodiment initializes the low-pass filter processing executed by the filter processing unit 42 (step 505). Specifically, the initialization of the low-pass filter in step 401 is executed by initializing the history of the posture discrimination probability value X used for the low-pass filter processing. In other words, on a right side of the above Formula (1), a first term “i=1 to P” is set to a “zero matrix”, and a second term “j=1 to Q” is set to a “zero matrix”. Further, in the posture discrimination unit 22 according to the present embodiment, the filter processing unit 42 executes the low-pass filter processing on the posture discrimination probability value X (see FIG. 5, step 107) calculated in the current period in an initialized state (step 506). The posture discrimination unit 22 according to the present embodiment determines the posture of the occupant 5 based on the posture discrimination probability value X′ after the low-pass filter processing executed in step 506, thereby discriminating that the posture PH is the “falling posture” (step 507).

That is, while the periods in which the erroneous discrimination of the “falling posture” is confirmed are consecutive, the posture PH of the occupant 5 discriminated in the period before the erroneous discrimination is first confirmed is maintained as the final output of the posture discrimination unit 22. Further, during this time, the history of the posture discrimination probability value X used for the low-pass filter processing is also not updated. As a result, the posture PH of the occupant 5 indicated in the final output of the posture discrimination unit 22 may deviate from the actual posture PH.

In view of this point, as described above, the posture discrimination unit 22 according to the present embodiment assumes that the actual posture PH has already changed to the “falling posture” when the number of consecutive times N of the periods in which the erroneous discrimination of the “falling posture” is confirmed reaches the consecutive threshold value Nth. That is, when the periods in which it is discriminated that the posture PH of the occupant 5 is the “falling posture” based on the falling discrimination probability value XC are consecutive for a certain degree, it is highly possible that the actual posture PH is changed to the “falling posture”. Further, by executing the low-pass filter processing in a state in which the history of the posture discrimination probability value X is initialized with respect to the posture discrimination probability value X calculated in the current period, the posture PH of the occupant 5 is discriminated to be the “falling posture” based on the falling discrimination probability value XC calculated in the current period. The control device 10 according to the present embodiment matches the posture PH of the occupant 5 indicated in the final output of the posture discrimination unit 22 with the actual posture PH.

Next, an advantage of the present embodiment will be described.

In the control device 10 having a function as the posture discrimination device 30, the skeleton points SP of the occupant 5 shown in the captured image Vd in the vehicle cabin 6 is detected, and the posture discrimination probability value X of the occupant 5 is calculated based on the detection of the skeleton points SP. The posture PH of the occupant 5 is discriminated based on the posture discrimination probability value X.

In addition, in the control device 10, as the posture discrimination probability value X, the falling discrimination probability value XC indicating the probability that the posture PH of the occupant 5 is the “falling posture” is calculated, and the change speed α of the falling discrimination probability value XC is calculated. When the change speed α of the falling discrimination probability value XC is equal to or greater than the predetermined speed threshold value α-th, it is determined that the erroneous discrimination may occur in the discrimination of the “falling posture” based on the falling discrimination probability value XC.

Next, an effect of the present embodiment will be described.

(1) The control device 10 as the posture discrimination device 30 includes the skeleton point detection unit 13 that detects the skeleton points SP of the person H included in the captured image Vd. In addition, the control device 10 includes the posture discrimination probability value calculation unit 21 that calculates the posture discrimination probability value X of the person H based on the detection of the skeleton points SP, and the posture discrimination unit 22 that discriminates the posture PH of the person H based on the posture discrimination probability value X. In addition, the posture discrimination probability value calculation unit 21 calculates, as the posture discrimination probability value X, the falling discrimination probability value XC indicating the probability that the posture PH of the person H is the “falling posture”. Further, the posture discrimination unit 22 includes the falling erroneous discrimination determination unit 41 that determines the erroneous discrimination of the “falling posture” based on the falling discrimination probability value XC. When the change speed α of the falling discrimination probability value XC is equal to or greater than the predetermined speed threshold value α-th, the falling erroneous discrimination determination unit 41 determines that the erroneous discrimination of the “falling posture” based on the falling discrimination probability value XC may occur.

That is, it takes some time for the posture PH of the person H to change from the “standing posture” to the “falling posture”. Therefore, if the change speed α of the falling discrimination probability value XC is too fast, it is highly possible that the value is the abnormal value. Therefore, it can be determined that the erroneous discrimination may occur in the discrimination of the “falling posture” based on the falling discrimination probability value XC.

Therefore, according to the above configuration, even in a situation in which the detection accuracy of the skeleton points SP based on a positional relationship with the camera 8 is lowered and thus the falling discrimination probability value XC cannot be correctly calculated, it is possible to promptly detect the possibility of occurrence of the erroneous discrimination of the “falling posture” based on the falling discrimination probability value XC. Therefore, it is possible to suppress the erroneous discrimination of the “falling posture” and accurately discriminate the posture PH of the person H.

(2) The posture discrimination probability value calculation unit 21 calculates the posture discrimination probability value X by inputting the feature amounts FV calculated based on the detection of the skeleton points SP into the inference model generated by the machine learning.

According to the above configuration, it is possible to accurately discriminate the posture PH of the person H based on the detection of the skeleton points SP by the image analysis. Further, there is an advantage that a posture of the person H can be discriminated by using only a two-dimensional image. Therefore, cost reduction can be achieved.

(3) The control device 10 periodically executes posture discrimination based on the detection of the skeleton points SP by the skeleton point detection unit 13, the posture discrimination probability value X obtained by the posture discrimination probability value calculation unit 21, and the posture discrimination probability value X obtained by the posture discrimination unit 22. In addition, the posture discrimination unit 22 includes the previous value storage unit 55 that stores the posture PH of the person H discriminated in the previous period, that is, the result of the posture discrimination in the previous period as the previous value PHb. The posture discrimination unit 22 sets the previous value PHb as the result of the posture discrimination in the current period when the erroneous discrimination of the “falling posture” is confirmed by the falling erroneous discrimination determination unit 41 based on the possibility of occurrence of the erroneous discrimination.

According to the above configuration, the posture PH of the person H can be determined so that the erroneous discrimination of the “falling posture” based on the falling discrimination probability value XC calculated in the current period does not become the final output of the posture discrimination. Therefore, it is possible to accurately discriminate the posture PH of the person H.

(4) When the number of consecutive times N of the periods in which the erroneous discrimination of the “falling posture” is confirmed reaches the predetermined consecutive threshold value Nth, the posture discrimination unit 22 discriminates that the posture PH of the person H is the “falling posture” regardless of the determination result of the falling erroneous discrimination determination unit 41.

That is, while the periods in which the erroneous discrimination of the “falling posture” is confirmed are consecutive, the posture PH of the person H discriminated in the period before the erroneous discrimination is first confirmed is maintained as the final output of the posture discrimination. As a result, the posture PH of the person H shown in the result of the posture discrimination may deviate from the actual posture PH. However, when the periods in which it is discriminated that the posture PH of the person H is the “falling posture” based on the falling discrimination probability value XC are consecutive for a certain degree, it is highly possible that the actual posture PH is changed to the “falling posture”. In such a case, by assuming that the actual posture PH has already changed to the “falling posture”, it is possible to match the posture PH of the person H indicated by the final output of the posture discrimination with the actual posture PH.

(5) The posture discrimination unit 22 stores the history of the posture discrimination probability value X calculated in or before the previous period. In addition, the posture discrimination unit 22 discriminates the posture PH of the person H based on the posture discrimination probability value X′ after the low-pass filter processing in which the history of the posture discrimination probability value X is reflected is executed with respect to the posture discrimination probability value X calculated in the current period. When the number of consecutive times N of the periods in which the erroneous discrimination of the “falling posture” is confirmed reaches the consecutive threshold value Nth, the posture discrimination unit 22 initializes the history of the posture discrimination probability value X used for the low-pass filter processing.

That is, by executing the low-pass filter processing in the state in which the history of the posture discrimination probability value X is initialized with respect to the posture discrimination probability value X calculated in the current period, the posture PH of the person H is discriminated to be the “falling posture” based on the falling discrimination probability value XC calculated in the current period. Therefore, the posture PH of the person H indicated by the final output of the posture discrimination can be matched with the actual posture PH that was assumed to have already changed to the “falling posture”.

(6) When the height β of the skeleton points SP shown in the captured image Vd is equal to or greater than the predetermined height threshold value β-th, the falling erroneous discrimination determination unit 41 determines that the erroneous discrimination of the “falling posture” based on the falling discrimination probability value XC may occur.

That is, when the posture PH of the person H changes from the “standing posture” to the “falling posture”, the height β of the skeleton points SP shown in the captured image Vd becomes low. Therefore, if the height β of the skeleton points SP is too high, it is highly possible that the value is an abnormal value.

Therefore, according to the above configuration, even in the situation in which the detection accuracy of the skeleton points SP is lowered based on the positional relationship with the camera 8 and thus the falling discrimination probability value XC cannot be correctly calculated, it is possible to promptly detect the possibility of occurrence of the erroneous discrimination of the “falling posture” based on the falling discrimination probability value XC. Therefore, it is possible to accurately discriminate the posture PH of the person H.

(7) When the falling erroneous discrimination determination unit 41 determines that the erroneous discrimination may occur based on the change speed α of the falling discrimination probability value XC and determines that the erroneous discrimination may occur based on the height β of the skeleton points SP, the falling erroneous discrimination determination unit 41 confirms the erroneous discrimination of the “falling posture” based on the falling discrimination probability value XC. Therefore, it is possible to accurately determine the erroneous discrimination of the “falling posture” based on the falling discrimination probability value XC.

(8) The posture discrimination unit 22 discriminates the posture PH of the occupant 5 located in the vehicle cabin 6 of the vehicle 1 as the posture PH of the person H.

According to the above configuration, the posture PH of the occupant 5 located in the vehicle cabin 6 of the vehicle 1 can be accurately discriminated while suppressing the erroneous discrimination of the “falling posture”. In particular, in the vehicle 1 in which the occupant 5 rides in the “standing posture” in which the discrimination of the “falling posture” is necessary, in many cases, the camera 8 is arranged at a position where the occupant 5 in the vehicle cabin 6 is captured from above, such as the ceiling portion 9. Therefore, when the occupant 5 moves below the camera 8, the image of the occupant 5 shown in the captured image Vd is likely to be in the state in which the lower body is hidden by the head or the upper body of the occupant 5. Further, the detection accuracy of the skeleton points SP is lowered, and thus the calculation accuracy of the posture discrimination probability value X is also lowered. For example, since the skeleton points SP of the person H included in the captured image Vd are densely detected, the falling discrimination probability value XC may increase. Therefore, a more remarkable effect can be obtained by performing the above falling erroneous discrimination determination for such a configuration.

The above embodiment can be modified and implemented as follows. The above embodiment and following modified examples can be implemented in combination with each other within a range in which no technically contradiction arises.

In the above embodiment, the feature amounts FV of the person H are calculated based on the position of the skeleton points SP included in the captured image Vd and the body dimensions indicated by the plurality of skeleton points SP. By inputting the feature amounts FV into the inference model generated by the machine learning, the posture discrimination probability value X of the person H shown in the captured image Vd is calculated. However, this disclosure is not limited to this, and may be applied to a configuration in which the posture discrimination probability value X is calculated based on the detection of the skeleton points SP by the image analysis by using a statistical method, a map calculation, or the like.

For example, the camera 8 may be optionally changed in a form by using an infrared camera or the like. Further, a position where the camera 8 is arranged may also be optionally changed. However, it is desirable to arrange the camera 8 in a high position such that an image of a whole body of the person H serving as the target person can be captured. Two or more cameras 8 may be used.

In addition, the skeleton points SP and the body dimensions used for the calculation of the posture discrimination probability value X may be optionally set.

In the above embodiment, the posture PH of the occupant 5 located in the vehicle cabin 6 of the vehicle 1 is discriminated as the posture PH of the person H shown in the captured image Vd. However, this disclosure is not limited to this, and may be applied to, for example, a configuration for discriminating the posture PH of the person H shown in the captured image Vd, on a road or in a building.

In the above embodiment, the “standing posture”, the “sitting posture”, and the “falling posture” are discriminated as the postures PH of the person H shown in the captured image Vd. The “standing posture” is further subdivided and discriminated into the “moving state”, the “still state”, and the “state of standing by using the hanging strap, the handrail, and the like”. However, this disclosure is not limited to this, and may be applied to, for example, a configuration in which the “sitting posture” is also subdivided and discriminated into more states, such as discriminating a plurality of states. For example, this disclosure may be applied to a configuration in which the number of discriminations is small, such as determining either the “standing posture” or the “falling posture”.

In the above embodiment, when it is determined that there is a possibility of the erroneous discrimination based on the change speed α of the falling discrimination probability value XC and it is determined that there is a possibility of the erroneous discrimination based on the height β of the skeleton points SP, the erroneous discrimination of the “falling posture” based on the falling discrimination probability value XC is confirmed. However, this disclosure is not limited to this, and may be applied to a configuration in which when it is determined that there is the possibility of the erroneous discrimination based on the change speed α of the falling discrimination probability value XC or it is determined that there is the possibility of the erroneous discrimination based on the height β of the skeleton points SP, the erroneous discrimination of the “falling posture” based on the falling discrimination probability value XC may be confirmed. Further, this disclosure may be applied to a configuration in which a result of falling erroneous discrimination determination by another method may be combined to confirm the erroneous discrimination of the “falling posture”. Furthermore, this disclosure may be applied to a configuration in which the erroneous discrimination of the “falling posture” is confirmed based on the change speed α of the falling discrimination probability value XC without executing the falling erroneous discrimination determination based on the heightβ of the skeleton points SP.

The speed threshold value α-th may be optionally changed. In addition, the height threshold value β-th may be optionally set. The predetermined skeleton points SPx for acquiring the height β of the skeleton points SP may be optionally set. Further, a method for acquiring the height β of the skeleton points SP may be changed. The consecutive threshold value Nth may also be optionally changed.

In the above embodiment, this disclosure includes the filter processing unit 42 that executes the low-pass filter processing for the posture discrimination probability value X, but may be applied to a configuration that does not include such a filter processing unit 42.

In the above embodiment, the posture discrimination probability value calculation unit 21 calculates the falling discrimination probability value XC indicating the probability that the posture PH of the person H is the “falling posture” as the posture discrimination probability value X. Further, the posture discrimination unit 22 includes, as an erroneous discrimination determination unit that determines the erroneous discrimination of the posture PH of the person H based on the posture discrimination probability value, the falling erroneous discrimination determination unit 41 that determines the erroneous discrimination of the “falling posture” based on the falling discrimination probability value XC. When the change speed α of the falling discrimination probability value XC is equal to or greater than the predetermined speed threshold value α-th, the falling erroneous discrimination determination unit 41 determines that the erroneous discrimination of the “falling posture” based on the falling discrimination probability value XC may occur.

However, this disclosure is not limited to this, and may be applied to a configuration in which the posture discrimination unit 22 includes an erroneous discrimination determination unit other than the falling erroneous discrimination determination unit 41. The erroneous discrimination determination unit may perform an erroneous discrimination determination of the posture PH of the person H based on the comparison between the change speed of the posture discrimination probability value X and a predetermined speed threshold value.

That is, the discrimination determination unit determines whether the change speed of the posture discrimination probability value X corresponding to the posture P of the person H is equal to or greater than the predetermined speed threshold value with respect to the posture PH of the person H other than the “falling posture”. When the change speed is equal to or higher than the predetermined speed threshold value, the discrimination determination unit may determine that the erroneous discrimination of the posture PH of the person H based on the posture discrimination probability value X may occur.

In this case, the posture PH of the person H for which the erroneous discrimination determination is performed may be optionally set. This disclosure may be applied to a configuration in which, for the postures PH of a plurality of persons H, the erroneous discrimination determination based on the comparison between the change speed of the posture discrimination probability value X and the predetermined speed threshold value is performed including the erroneous discrimination of the “falling posture”.

In addition, a handling of a case where the erroneous discrimination for the posture PH of the person H is confirmed or a case where the number of consecutive times of the periods in which the erroneous discrimination is confirmed reaches the consecutive threshold value, or the use of the low-pass filter may be similarly applied to the posture discrimination other than the “falling posture”.

A posture discrimination device includes: a skeleton point detection unit configured to detect a skeleton point of a person included in a captured image; a posture discrimination probability value calculation unit configured to calculate a posture discrimination probability value of the person based on detection of the skeleton point; and a posture discrimination unit configured to discriminate a posture of the person based on the posture discrimination probability value. The posture discrimination probability value calculation unit calculates, as the posture discrimination probability value, a falling discrimination probability value indicating a probability that the posture of the person is a falling posture. The posture discrimination unit includes a falling erroneous discrimination determination unit configured to determine an erroneous discrimination of the falling posture based on the falling discrimination probability value. The falling erroneous discrimination determination unit determines that there is a possibility of occurrence of the erroneous discrimination when a change speed of the falling discrimination probability value is equal to or higher than a predetermined speed threshold value.

That is, it takes some time for the posture of the person to change from a “standing posture” to the “falling posture”. Therefore, if the change speed of the falling discrimination probability value is too fast, it is highly possible that the value is an abnormal value. Therefore, it can be determined that the erroneous discrimination may occur in the discrimination of the “falling posture” based on the falling discrimination probability value.

Therefore, according to the above configuration, even in a situation in which a detection accuracy of the skeleton point is lowered based on a positional relationship with a camera and thus the falling discrimination probability value cannot be correctly calculated, it is possible to promptly detect the possibility of occurrence of the erroneous discrimination of the “falling posture” based on the falling discrimination probability value. Therefore, it is possible to accurately discriminate the posture of the person by suppressing the erroneous discrimination of the “falling posture”.

It is preferable that in the posture discrimination device, the posture discrimination probability value calculation unit calculates the posture discrimination probability value by inputting a feature amount calculated based on the detection of the skeleton point into an inference model generated by machine learning.

According to the above configuration, it is possible to accurately discriminate the posture of the person based on the detection of the skeleton point by image analysis. Further, there is an advantage that the posture of the person can be discriminated by using only a two-dimensional image. Therefore, cost reduction can be achieved.

It is preferable that in the posture discrimination device, the detection of the skeleton point, calculation of the posture discrimination probability value, and the posture discrimination based on the posture discrimination probability value are periodically executed. The posture discrimination device further includes a previous value storage unit configured to store a result of a posture discrimination in a previous period as a previous value. When the erroneous discrimination of the falling posture is confirmed based on the possibility of the occurrence of the erroneous discrimination, the posture discrimination unit sets the previous value as a result of the posture discrimination in a current period.

According to the above configuration, the posture of the person can be determined so that the erroneous discrimination of the “falling posture” based on the falling discrimination probability value calculated in the current period does not become a final output of the posture discrimination. Therefore, it is possible to accurately discriminate the posture of the person.

It is preferable that in the posture discrimination device, the posture discrimination unit is configured to discriminate that the posture is the falling posture regardless of a determination result of the falling erroneous discrimination determination unit in a case where the number of consecutive times of periods in which the erroneous discrimination of the falling posture is confirmed reaches a predetermined consecutive threshold value.

That is, while the periods in which the erroneous discrimination of the “falling posture” is confirmed are consecutive, the posture of the person discriminated in a period before the erroneous discrimination is first confirmed is maintained as the final output of the posture discrimination. As a result, the posture of the person shown in the result of the posture discrimination may deviate from an actual posture. However, when the periods in which it is discriminated that the posture of the person is the “falling posture” based on the falling discrimination probability value are consecutive for a certain degree, it is highly possible that the actual posture is changed to the “falling posture”. Then, in such a case, it is assumed that the actual posture has already changed to the “falling posture”, so that the posture indicated by the final output of the posture discrimination can be matched with the actual posture.

It is preferable that in the posture discrimination device, the posture discrimination unit stores a history of the posture discrimination probability value calculated in or before the previous period, and discriminates the posture of the person based on the posture discrimination probability value after a low-pass filter processing that reflects the history of the posture discrimination probability value is executed with respect to the posture discrimination probability value calculated in the current period, and when the number of consecutive times of the periods in which the erroneous discrimination of the falling posture is confirmed reaches the consecutive threshold value, the history of the posture discrimination probability value used for the low-pass filter processing is initialized.

That is, by executing the low-pass filter processing in a state in which the history of the posture discrimination probability value is initialized with respect to the posture discrimination probability value calculated in the current period, the posture of the person is discriminated to be the “falling posture” based on the falling discrimination probability value calculated in the current period. Therefore, the posture of the person indicated by the final output of the posture discrimination can be matched with the actual posture that was assumed to have already changed to the “falling posture”.

It is preferable that in the posture discrimination device, the falling erroneous discrimination determination unit determines that there is the possibility of the occurrence of the erroneous discrimination when a height of the skeleton point shown in the captured image is equal to or higher than a predetermined height threshold value.

That is, when the posture of the person changes from the “standing posture” to the “falling posture”, the height of the skeleton point shown in the captured image is low. Therefore, if the height of the skeleton point is too high, it is highly possible that the value is an abnormal value.

Therefore, according to the above configuration, even in the situation in which the detection accuracy of the skeleton point is lowered based on the positional relationship with the camera and thus the falling discrimination probability value cannot be correctly calculated, it is possible to promptly detect the possibility of occurrence of the erroneous discrimination of the “falling posture” based on the falling discrimination probability value. Therefore, it is possible to accurately discriminate the posture of the person.

It is preferable that in the posture discrimination device, the falling erroneous discrimination determination unit confirms, when it is determined that there is the possibility of the occurrence of the erroneous discrimination based on the change speed of the falling discrimination probability value, and there is the possibility of the occurrence of the erroneous discrimination based on the height of the skeleton point, the erroneous discrimination of the falling posture.

According to the above configuration, it is possible to accurately determine the erroneous discrimination of the “falling posture” based on the falling discrimination probability value.

It is preferable that in the posture discrimination device, the posture discrimination unit discriminates a posture of an occupant located in a vehicle cabin of a vehicle as the posture of the person.

According to the above configuration, the posture of the occupant located in the vehicle cabin of the vehicle can be accurately discriminated while suppressing the erroneous discrimination of the “falling posture”. In particular, in the vehicle in which the occupant rides in the “standing posture” in which the discrimination of the “falling posture” is necessary, in many cases, the camera is arranged at a position where an image of the occupant in the vehicle cabin is captured from above, such as a ceiling portion. Therefore, when the occupant moves below the camera, an image of the occupant shown in the captured image is likely to be in a state in which a lower body is hidden by a head or an upper body of the occupant. Therefore, the detection accuracy of the skeleton point is lowered, and thus a calculation accuracy of the posture discrimination probability value is also lowered. For example, if the skeleton points of the person included in the captured image are densely detected, the falling discrimination probability value may increase. Therefore, a more remarkable effect can be obtained by executing a falling erroneous discrimination determination of each of the above configurations for such a configuration.

A posture discrimination device includes: a skeleton point detection unit configured to detect a skeleton point of a person included in a captured image; a posture discrimination probability value calculation unit configured to calculate a posture discrimination probability value of the person based on detection of the skeleton point; and a posture discrimination unit configured to discriminate a posture of the person based on the posture discrimination probability value. The posture discrimination unit includes an erroneous discrimination determination unit configured to determine an erroneous discrimination of the posture of the person based on the posture discrimination probability value. The erroneous discrimination determination unit determines that there is a possibility of occurrence of the erroneous discrimination when a change speed of the posture discrimination probability value is equal to or higher than a predetermined speed threshold value.

That is, it takes some time for the posture of the person to change. Therefore, if the change speed of the posture discrimination probability value is too fast, it is highly possible that the value is an abnormal value. Therefore, it can be determined that the erroneous discrimination may occur in the discrimination of the “posture of the person” based on the posture discrimination probability value.

Therefore, according to the above configuration, even in a situation in which a detection accuracy of the skeleton point is lowered based on a positional relationship with a camera and thus the falling discrimination probability value cannot be correctly calculated, it is possible to promptly detect the possibility of occurrence of the erroneous discrimination of the “posture of the person” based on the posture discrimination probability value. Therefore, it is possible to accurately discriminate the posture of the person.

According to this disclosure, it is possible to accurately discriminate the posture of the person by suppressing the erroneous discrimination.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A posture discrimination device comprising:
a skeleton point detection unit configured to detect a skeleton point of a person included in a captured image;
a posture discrimination probability value calculation unit configured to calculate a posture discrimination probability value of the person based on detection of the skeleton point; and
a posture discrimination unit configured to discriminate a posture of the person based on the posture discrimination probability value, wherein
the posture discrimination probability value calculation unit calculates, as the posture discrimination probability value, a falling discrimination probability value indicating a probability that the posture of the person is a falling posture,
the posture discrimination unit includes a falling erroneous discrimination determination unit configured to determine an erroneous discrimination of the falling posture based on the falling discrimination probability value, and
the falling erroneous discrimination determination unit determines that there is a possibility of occurrence of the erroneous discrimination when a change speed of the falling discrimination probability value is equal to or higher than a predetermined speed threshold value.

2. The posture discrimination device according to claim 1, wherein
the posture discrimination probability value calculation unit calculates the posture discrimination probability value by inputting a feature amount calculated based on the detection of the skeleton point into an inference model generated by machine learning.

3. The posture discrimination device according to claim 1, wherein
the detection of the skeleton point, calculation of the posture discrimination probability value, and the posture discrimination based on the posture discrimination probability value are periodically executed,
the posture discrimination device further comprises a previous value storage unit configured to store a result of a posture discrimination in a previous period as a previous value, and
when the erroneous discrimination of the falling posture is confirmed based on the possibility of the occurrence of the erroneous discrimination, the posture discrimination unit sets the previous value as a result of the posture discrimination in a current period.

4. The posture discrimination device according to claim 3, wherein
the posture discrimination unit is configured to discriminate that the posture is the falling posture regardless of a determination result of the falling erroneous discrimination determination unit in a case where the number of consecutive times of periods in which the erroneous discrimination of the falling posture is confirmed reaches a predetermined consecutive threshold value.

5. The posture discrimination device according to claim 4, wherein
the posture discrimination unit stores a history of the posture discrimination probability value calculated in or before the previous period, and discriminates the posture of the person based on the posture discrimination probability value after a low-pass filter processing that reflects the history of the posture discrimination probability value is executed with respect to the posture discrimination probability value calculated in the current period, and
when the number of consecutive times of the periods in which the erroneous discrimination of the falling posture is confirmed reaches the consecutive threshold value, the history of the posture discrimination probability value used for the low-pass filter processing is initialized.

6. The posture discrimination device according to claim 1, wherein
the falling erroneous discrimination determination unit determines that there is the possibility of the occurrence of the erroneous discrimination when a height of the skeleton point shown in the captured image is equal to or higher than a predetermined height threshold value.

7. The posture discrimination device according to claim 6, wherein
the falling erroneous discrimination determination unit confirms, when it is determined that there is the possibility of the occurrence of the erroneous discrimination based on the change speed of the falling discrimination probability value, and there is the possibility of the occurrence of the erroneous discrimination based on the height of the skeleton point, the erroneous discrimination of the falling posture.

8. The posture discrimination device according to claim 1, wherein
the posture discrimination unit discriminates a posture of an occupant located in a vehicle cabin of a vehicle as the posture of the person.

9. A posture discrimination device comprising:
a skeleton point detection unit configured to detect a skeleton point of a person included in a captured image;
a posture discrimination probability value calculation unit configured to calculate a posture discrimination probability value of the person based on detection of the skeleton point; and
a posture discrimination unit configured to discriminate a posture of the person based on the posture discrimination probability value, wherein
the posture discrimination unit includes an erroneous discrimination determination unit configured to determine an erroneous discrimination of the posture of the person based on the posture discrimination probability value, and
the erroneous discrimination determination unit determines that there is a possibility of occurrence of the erroneous discrimination when a change speed of the posture discrimination probability value is equal to or higher than a predetermined speed threshold value.
